(19) United States
(12) Patent Application Publication
(10) Pub. No.: US 2022/0208318 A1
(43) Pub. Date: Jun. 30, 2022

(54) PATIENT MOVEMENT DETECTION AND COMMUNICATION

(71) Applicant: Hill-Rom Services, Inc., Batesville, IN (US)

(72) Inventors: Sinan Batman, Pittsford, NY (US); Karrie Browne, Cary, NC (US); Michael Hood, Batesville, IN (US); Susan Kayser, Batesville, IN (US); Georg Köllner, Saalfeld (DE); Dee Kumpar, Saginaw, MI (US); Mary Markham-Feagins, Indianapolis, IN (US); Dana Peco, Sarasota, FL (US); Mary L. Pfeffer, Charleston, SC (US); Kelli F. Rempel, Chapel Hill, NC (US); Eugene Urrutia, Apex, NC (US); Neal Wiggermann, Batesville, IN (US); Lori Ann Zapfe, Milroy, IN (US)

(21) Appl. No.: 17/457,575

(22) Filed: Dec. 3, 2021

Related U.S. Application Data
(60) Provisional application No. 63/131,821, filed on Dec. 30, 2020.

(51) Int. Cl. G16H 10/60; G16H 40/63

(52) U.S. Cl. CPC G16H 10/60; G16H 40/63

(57) ABSTRACT

A mobility management system receives movement data from sensors that detect movements of a patient resting in a patient support apparatus and movement data from one or more mobility detection devices that detect movement of the patient when the patient exits the patient support apparatus. The system determines a mobility status based on at least the movement data received from the patient support apparatus and the one or more mobility detection devices, and adjusts a care protocol for the patient based on the mobility status.

### BACKGROUND

A patient's mobility can determine whether the patient is at risk for falls, infections, pressure injuries, and other adverse conditions. Accordingly, a patient's mobility typically impacts the equipment, staffing, and care protocols that are selected for the patient within a healthcare facility such as a hospital. Additionally, a patient's mobility can help determine whether the patient can be safely discharged from the healthcare facility.

Undetected changes in a patient's mobility status are common. Also, patient mobility changes, even when detected, are often not communicated across all shifts, departments, and caregivers responsible for treating and providing care to the patient. Undetected or uncommunicated changes in the patient's mobility status can delay the patient from receiving appropriate care protocols, physical therapy, and discharge planning.

### SUMMARY

In general terms, the present disclosure relates to detecting and communicating changes in patient mobility to drive patient care. The detected changes in patient mobility are communicated to caregivers responsible for providing care to the patient. Various aspects are described in this disclosure, which include, but are not limited to, the following aspects.

One aspect relates to a mobility management system comprising: a controller having at least one processing device, and at least one computer readable data storage device storing software instructions that, when executed by the at least one processing device, cause the controller to: receive movement data from sensors that detect movements of a patient resting in a patient support apparatus; receive movement data from one or more mobility detection devices that detect movement of the patient when the patient exits the patient support apparatus; determine a mobility status based on at least the movement data received from the patient support apparatus and the one or more mobility detection devices; and adjust a care protocol for the patient based on the mobility status.

Another aspect relates to a method of determining a mobility status of a patient, the method comprising: receiving a first set of movement data from sensors on a patient support apparatus; receiving a second set of movement data from one or more mobility detection devices located within a patient environment around the patient support apparatus; determining a mobility status based on the first and second sets of movement data; comparing the mobility status to a prior mobility status; and communicating the mobility status to a mobile device.

Another aspect relates to a computer-readable data storage medium comprising software instructions that, when executed, cause at least one computing device to: receive a first set of movement data from sensors on a patient support apparatus; receive a second set of movement data from one or more mobility detection devices located within a patient environment around the patient support apparatus; determine a mobility status based on the first and second sets of movement data; compare the mobility status to a prior mobility status; and communicate the mobility status to a mobile device.

A variety of additional aspects will be set forth in the description that follows. The aspects can relate to individual features and to combination of features. It is to be understood that both the foregoing general description and the following detailed description are exemplary and explanatory only and are not restrictive of the broad inventive concepts upon which the embodiments disclosed herein are based.

### DESCRIPTION OF THE FIGURES

The following drawing figures, which form a part of this application, are illustrative of the described technology and are not meant to limit the scope of the disclosure in any manner.

FIG. 1 schematically illustrates a mobility management system.

FIG. 2 illustrates an example patient support apparatus included in the mobility management system of FIG. 1.

FIG. 3 illustrates a method of monitoring and communicating a mobility status of a patient using the mobility management system of FIG. 1.

FIG. 4 illustrates a user interface generated on a mobile device of the mobility management system of FIG. 1.

FIG. 5 illustrates another user interface generated on a mobile device of the mobility management system of FIG. 1.

FIG. 6 illustrates another user interface generated on a mobile device of the mobility management system of FIG. 1.

FIG. 7 illustrates a method of adjusting a care protocol for a patient based on a changed mobility status of the patient using the mobility management system of FIG. 1.

FIG. 8 illustrates a method of turning a patient to mitigate pressure injuries using the mobility management system of FIG. 1.

FIG. 9 illustrates an example of a walk assist device of the mobility management system of FIG. 1.

FIG. 10 schematically illustrates an example controller of the mobility management system of FIG. 1.

### DETAILED DESCRIPTION

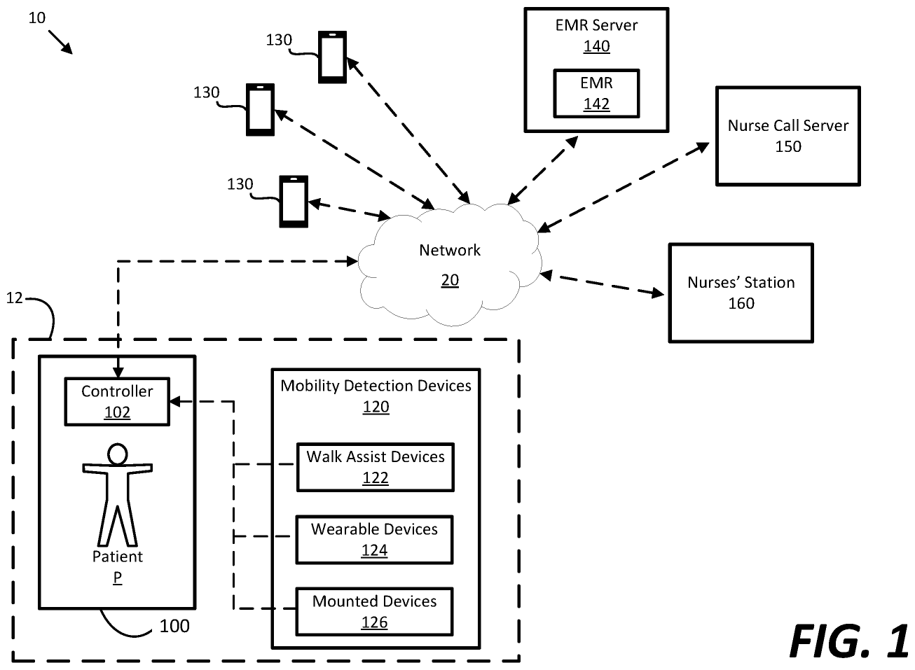

FIG. 1 schematically illustrates a mobility management system 10 that detects and trends the mobility of a patient P within a patient environment 12. The mobility management system 10 determines a mobility status of the patient P in real-time, and communicates the mobility status of the patient P in an easily understood format. The mobility status can be used by caregivers to guide care protocols for the patient P to address at least pressure injury prevention, fall prevention, physical therapy planning, and discharge planning.

In some illustrative examples, the patient environment 12 is a patient room within a healthcare facility such as a hospital, a surgical center, a nursing home, a long term care facility, and the like. In other examples, the patient environment 12 is the patient P′s home.

The mobility management system 10 includes a network 20 that receives a mobility status of the patient P that is based on movement data received from a plurality of sensors within the patient environment 12. The mobility status can be calculated or scaled to represent as a mobility score such as a bedside mobility assessment tool (BMAT) score, an activity measure for post-acute care (AM-PAC) score, or a proprietary mobility score. The mobility score can be based on at least the movement data and patient demographics and physiological data acquired from an electronic medical record (EMR) 142 (alternatively termed electronic health record (EHR)) of the patient P. The EMR 142 is stored in an EMR server 140. The network 20 communicates trends in the patient P′s mobility status to drive care activity for the patient P.

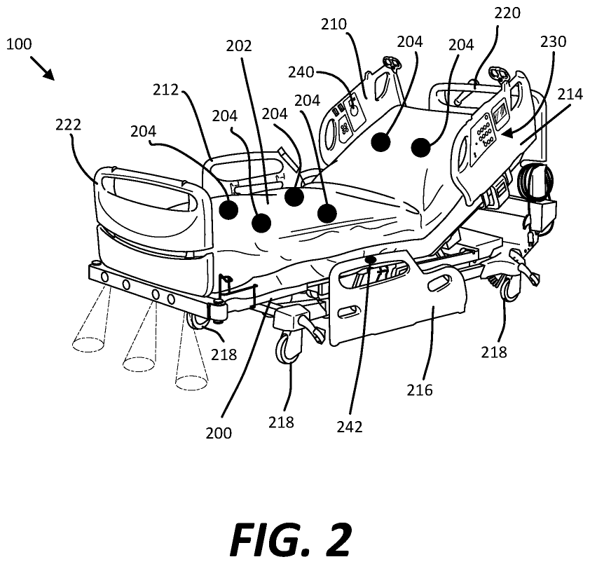

As shown in FIG. 1, the patient environment 12 includes a patient support apparatus 100 on which the patient P can rest. In certain examples, the patient support apparatus 100 is a bed such as the one that is shown and described in more detail below with reference to the example shown in FIG. 2. Alternatively, the patient support apparatus 100 can be a chair, a recliner, a stretcher, surgical table, or any other apparatus on which a patient can rest. As will be described in more detail, the patient support apparatus 100 includes sensors that detect movements of the patient P while the patient P rests on the patient support apparatus 100.

The patient environment 12 further includes mobility detection devices 120 that each includes one or more sensors that detect the movements of the patient P when the patient P is not resting on the patient support apparatus 100. The mobility detection devices 120 include walk assist devices 122, wearable devices 124, and mounted devices 126.

Each mobility detection device 120 communicates the detected movements of the patient P to a controller 102. In the example illustrated in FIG. 1, the controller 102 is a component of the patient support apparatus 100, and may also be used to control the operation of the patient support apparatus. In alternative examples, the controller 102 can be a standalone device external from the patient support apparatus 100. In yet further examples, the controller 102 can be a component of another device or apparatus located in the patient environment 12.

In addition to detecting movements of the patient P, each mobility detection device 120 can also detect and transmit additional parameters to the controller 102 including patient vital signs such as heart rate, respiration rate, SpO2, and non-invasive blood pressure. The detected movements and additional parameters of the patient P are used by the controller 102 to determine a variety of clinical assessments and scores, including a Braden's mobility score, sepsis prediction score, a pressure injury risk score, a falls risk score, and the like.

The controller 102 is a centralized analytics computation device within the mobility management system 10. The controller 102 aggregates the movements detected by the mobility detection devices 120 with the movements detected by the patient support apparatus 100. Advantageously, the controller 102 can reduce the amount of data that is transmitted across a local area network of a healthcare facility, and can also reduce the amount of data that is processed and analyzed by on-premises or edge-servers of the healthcare facility, and thereby reduce the strain on the bandwidth of the local area network and on-premises servers. The controller 102 can also improve the data upload bandwidth and reduce network lags for cloud-based servers utilized by the healthcare facility to perform data processing and analytics.

One or more wireless communications protocols including Wi-Fi, Bluetooth, Z-Wave, Zigbee, and the like, and/or one or more wired communications protocols including Ethernet, USB, and like can be used to connect the sensors of the patient support apparatus 100 and mobility detection devices 120 to the controller 102. These communications protocols provide an Internet of Things (IOT) network within the patient environment 12 that is private and secure. This can improve communication among the devices located within the patient environment 12, to authenticate, authorize, and associate the devices with each other, and perform coordinated operations. These functionalities, including authentication and authorization, can be distributed and performed in the IOT network with or without using a dedicated edge-computational resources of the healthcare facility.

Additionally, each of the patient support apparatus 100 and mobility detection devices 120 can act as a communication and computation network node within the IOT network. The functionalities can be localized at the controller 102 or distributed among network nodes using or completely avoiding an edge or cloud based computational infrastructure.

The IOT network enables the controller 102 to locally and remotely monitor and control each or some of the mobility detection devices 120 and the patient support apparatus 100 through device or caregiver issued instructions. Additionally, the controller 102 allows sharing of measurements to perform a risk score calculation that may require collection of data from multiple devices within the patient environment 12 including the mobility detection devices 120 and the patient support apparatus 100, as well as data acquired from the EMR 142.

Still referring to FIG. 1, the walk assist devices 122 are devices that the patient P can use to help maintain their balance and stability as they walk or otherwise move around the patient environment 12. Examples of walk assist device 122 include, without limitation, a walker, a walking cane, a bathroom transportation aide, a patient lift, and similar devices.

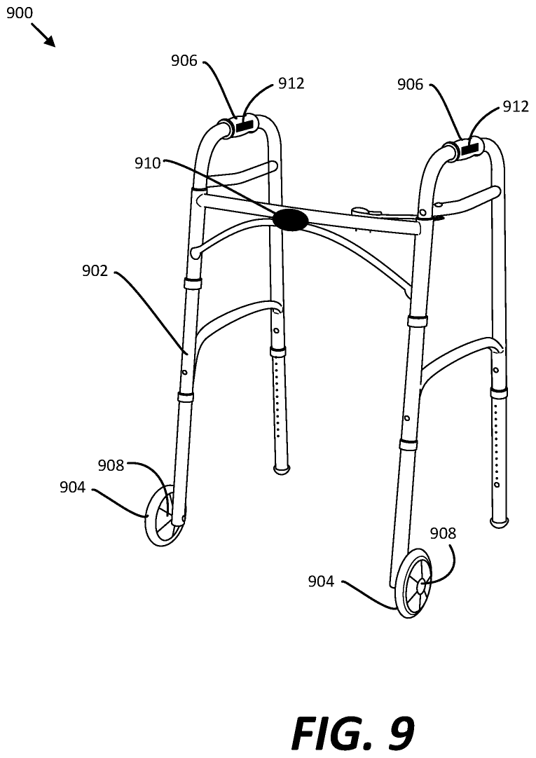

FIG. 9 illustrates a walker 900 that is an example of a walk assist device shown in FIG. 1. The walker 900 can be used in a healthcare facility or in the patient P′s home to transmit movement data and alerts to the controller 102 of the patient support apparatus 100. The walker 900 can utilize Wi-Fi, Zigbee, cellular networks, and other wireless technologies and Internet of Things (JOT) communication standards to send the movement data and alerts to the controller 102. The controller 102 can forward the alerts to the network 20.

The walker 900 has a lightweight frame 902 and one or more wheels 904 attached the frame. The walker 900 further includes handles 906 that are gripped by the patient P when using the walker 900 to maintain their balance and stability while walking.

The walker 900 can include one or more sensors 908 connected to the wheels 904 to detect rotation of the wheels 904 to measure the patient P′s frequency of use, gait, speed, acceleration, number of steps, equilibrium, wait times between movements, falls or near falls, and other measurements. Alternatively, or in addition to the sensors 908, the walker 900 may also have additional sensors 910 such as a GPS sensor, accelerometer, wireless real-time locating system (RTLS) tag, and similar sensors to track the patient P′s mobility. A GPS sensor can be especially helpful to track the location of a patient diagnosed with Alzheimer's such as when the patient leaves the patient environment 12 without assistance from a caregiver or family member.

The walker 900 can further include one or more sensors 912 connected to the handles 906 that measure additional parameters such as the patient P′s equilibrium or balance and one or more physiological variables of the patient P while using the walker 900. Examples of the physiological variables that can be measured from the sensors 912 include heart rate, pulse, and SpO₂. The sensors 912 can also measure the patient P′s strength when gripping the handles 906 to detect whether there is a change in grip showing patient fatigue, weakness, or deterioration.

In certain examples, the walker 900 triggers an alert containing an alert type and a device location when the patient P experiences a lack of equilibrium, a fall, a pause between movements that exceeds a threshold, or leaves a designated area such as the patient environment 12 without assistance from a caregiver. The walker 900 wirelessly sends the alert to the controller 102 of the patient support apparatus 100, and the controller 102 can forward the alert to the network 20. The network 20 can then transmit the alert to at least one of the mobile devices 130, EMR server 140, nurse call server 150, and/or nurses' station 160.

The walker 900 can include self-charging capability, such as from transforming the kinetic energy from the rolling of the wheels 904 into electrical energy to charge the onboard electronics. Additionally, the walker 900 can include charger plugs or be configured for in-place charging when parked on top of a charging base to charge the onboard electronics when the walker 900 is not in use. Typical daily operation of the walker 900 can be sufficient for the walker 900 to operate without requiring charging from the charger plugs or in-place charging. However, the walker 900 can remain charged for a predetermine period of time (e.g., one week) while remaining idle. The walker 900 can operate like a regular walker when uncharged.

The wearable devices 124 are motion sensors attached to the patient P′s body to detect the patient P′s mobility. In some instances, the motion sensors are attached directly to the patient P′s body such as in a body patch. In other examples, the motion sensors are embedded in clothing worn by the patient P such as socks or in an accessory worn by the patient P such as a wristwatch, a bracelet, an ankle band, or a chest band. The wearable devices 124 can include GPS sensors, accelerometers, wireless real-time locating system (RTLS) tags, and similar devices to track the patient P′s mobility. As discussed above, a GPS sensor can be especially helpful to track the location of a patient diagnosed with Alzheimer's.

Like the walker 900, the wearable devices 124 transmit movement data detected from the patient P to the controller 102 of the patient support apparatus 100. The wearable devices 124 can utilize Wi-Fi, Zigbee, and other wireless technologies and Internet of Things (IOT) communication standards to send the movement data to the controller 102.

In some examples, the wearable devices 124 are part of a real-time locating system (RTLS) that can be used to detect and track the patient P′s movements. In some instances, the wearable devices 124 when used together with the patient support apparatus 100, walk assist devices 122, and mounted devices 126 can increase the spatial resolution of the RTLS.

The mounted devices 126 include devices that are mounted to a wall, a ceiling, or to another device within the patient environment 12 such as the patient support apparatus 100, a digital whiteboard, a portable stand, a vital sign monitor, a chair, and the like. The mounted devices 126 can include one or more video cameras or radar transceivers that can be used to detect and track the patient P′s movements within the patient environment 12.

One or more algorithms stored on a memory of the controller 102 when executed by the processor of the controller 102 cause the controller 102 to process the aggregated movement data collected from the patient support apparatus 100 and mobility detection devices 120 to determine the mobility status of the patient P within the patient environment 12. The controller 102 then transfers the mobility status of the patient P to the network 20.

Additionally, the controller 102 can automatically document patient P′s mobility including transitions from the patient support apparatus 100 to a chair within the patient environment 12, from the patient support apparatus 100 to the walker 900 to the chair in the patient environment 12, time spent outside the patient support apparatus 100, time spent in the chair, time spent outside of the patient environment 12, number of steps per day, maximum length of walk and daily walking trends, and the like. The controller 102 can distinguish between unassisted motions and caregiver-assisted motions through sensor readings on the patient support apparatus 100 and mobility detection devices 120, and other devices within the patient environment 12 including RTLS devices that can determine patient to caregiver proximity.

In alternative examples, controller 102 transfers the aggregated movement data to an external server connected to the network 20. Upon receipt of the aggregated movement data, one or more algorithms stored on a memory of the external server, when executed by a processor of the external server, cause the external server to process the aggregated movement data to determine the mobility status of the patient P. The external server can be an on-premises or edge-server located within the healthcare facility, or can be a cloud-based server.

The network 20 can communicate changes in the patient P′s mobility status directly to a plurality of mobile devices 130. A mobile device 130 can be carried by a caregiver in the healthcare facility. Also, a mobile device 130 can be carried by a family member of the patient P. An application installed on each mobile device 130 allows each caregiver and/or family member to use his or her mobile device 130 to monitor alerts and notifications regarding the mobility status of the patient P, and to conduct voice and video communications with the patient P. In some examples, the alerts are disabled when the controller 102 detects the patient P is sleeping.

The network 20 can also communicate changes in the patient P′s mobility status directly to a nurses' station 160 which is an area in a healthcare facility, such as a hospital or nursing home, where caregivers such as nurses' and other staff work when not working directly with the patient P such as where they can perform administrative tasks. The nurses' station 160 includes one or more computing devices connected to the network 20.

The network 20 can also communicate the changes in the patient P′s mobility status to a nurse call server 150 that manages communications sent between the mobile devices 130, as well as communications between the mobile devices 130 and the nurses' station 160.

The controller 102, by using the network 20, can compare a current mobility status of the patient P against a prior mobility assessment of the patient P stored in the EMR 142. The comparison determines whether the current mobility status of the patient P matches the prior mobility assessment stored in the EMR 142. An inconsistency in the EMR 142 may result from a recent change in the patient P′s mobility status that has not yet been updated in the EMR 142 or may result from the prior mobility assessment stored in the EMR 142 being incorrect.

When the controller 102 determines that an inconsistency exists, the controller 102 can generate an alert that can be sent to the caregivers via the network 20. The controller 102 can provide the caregivers with the updated mobility status of the patient P. In some examples, the controller 102 automatically updates the mobility assessment of the patient P stored in the EMR 142. Alternatively, the controller 102 can prompt a caregiver to validate the updated mobility assessment of the patient P before storing the updated mobility assessment in the EMR 142.

The controller 102 can also help identify functional decline and pressure injury risk for the patient P. For example, the controller 102 can track the amount of time that the patient P has not left the patient support apparatus 100 and can alert a caregiver or family member when the amount of time exceeds a threshold set by a physician of the healthcare facility, the caregiver, or the family member. The controller 102 can also track the amount of time that the patient P has not moved substantially in the patient support apparatus 100.

As a further example, when the patient P has a prior mobility assessment indicating that the patient P is mobile, and the controller 102 detects from the aggregated movement data that the patient P has not left the patient support apparatus 100 after a predetermined period of time (e.g., 24 hours), the controller 102 can send an alert to a caregiver or family member. When the controller 102 detects that the patient P′s mobility has declined or is trending in a manner at odds with the patient P′s discharge plan, the controller 102 can send an alert to the caregiver or family member. The controller 102 can order physical therapy, or provide a recommendation for physical therapy, when a decline in mobility for the patient P is detected.

Also, the controller 102 can recommend ordering a walk assist device such as a walker or walking cane for the patient P or suggest an alternative care pathway based on the patient P′s mobility. Additionally, the controller 102 can generate an advanced mobility report on timing of patient turns, frequency of patient turns, whether the patient turns are assisted or not, time spent in each position, and body and extremity (major and minor) movement statistics.

In the perioperative context, the controller 102 can alert staff that the patient P needs to be moved before surgery while in a PreOp area. The position of the patient P and cumulative time on a surface such as the patient support apparatus 100 across the PreOp, intra-op, and PostOp areas can be communicated by the controller 102 via the network 20 to the next level of care within a surgical unit or after discharge from PostOp, as well as to nursing managers to provide suggestions on adjusting the support angle and mattress firmness of the patient support apparatus 100, as well as other surface changes, next best caregiver action, and the like.

The same detection and monitoring of the patient P′s movement in the patient support apparatus 100 can be performed at the patient P′s home to inform a family member that the patient P has not left the bed or has not been turned after a predetermined period of time. This can help reduce pressure injury risk and identify patient decline when the patient P is home.

Additionally, the walker 900, when used in the patient P′s home, can transmit movement data to an external application for tracking the patient P′s movements while in the patient P′s home. The external application can be used by the patient P′s physician to monitor the patient P′s progress and/or recovery, and to compare movement trends between the healthcare facility where the patient P was admitted and patient P′s home, and vice versa.

FIG. 2 illustrates an example of the patient support apparatus 100 that can be included in the patient environment 12 of FIG. 1. While FIG. 2 depicts the patient support apparatus 100 as a hospital bed, alternative examples are possible where the patient support apparatus 100 is a chair, a recliner, surgical table, or any other type of support apparatus. Accordingly, the description provided herein is not limited to hospital beds.

The patient support apparatus 100 includes a frame 200 that supports a mattress 202. The mattress 202 is flexible and conforms to the profile of the frame 200 as the orientation of the frame 200 is adjusted between horizontal and upright orientations. The mattress 202 includes one or more bladders that can be inflated and deflated to adjust the firmness of the mattress.

The patient support apparatus 100 includes sensors 204 that detect movements of the patient P while the patient P rests on the patient support apparatus 100. The sensors 204 can include load cells, such as piezoelectric sensors, that produce a voltage or current signal indicative of a weight impressed on the load cell from the patient P′s body. The sensors 204 can also include pressure sensors that measure a resistance inversely proportional to the pressure applied on the sensors from the weight of the patient P′s body. The patient support apparatus 100 can also include a variety of other types of sensors, including capacitance sensors, to detect the movement of the patient P while the patient P rests on the patient support apparatus 100.

The sensors 204 are mounted on the frame 200 and are positioned under the mattress 206. Also, the sensors 204 can be coupled to a top or bottom surface of the mattress 202, or can be positioned within an interior region of the mattress 202.

The frame 200 includes a left siderail assembly having at least one left siderail mounted on the left side of the frame and a right siderail assembly having at least one right siderail mounted on the right side of the frame. In example depicted in FIG. 2, the left siderail assembly includes an upper left siderail 210 and a lower left siderail 212, and the right siderail assembly includes an upper right siderail 214 and a lower right siderail 216.

Each siderail 210-216 is positionable at a deployed position at which its upper edge is higher than the top of the mattress 202 and at a stowed position at which its upper edge is lower than the top of the mattress 202. When the deployed position, a siderail prevents the patient P from exiting the patient support apparatus 100. When in the stowed position, a siderail allows the patient P to enter and exit the patient support apparatus 100. In the example embodiment illustrated in FIG. 2, the upper left siderail 210, lower left siderail 212, and upper right siderail 214 are in the deployed position, and the lower right siderail 216 is in the stowed position.

The patient support apparatus 100 further includes wheels 218 to facilitate the portability of the patient support apparatus 100, and a headboard 220 and a footboard 222. In certain embodiments, the footboard 222 is removable from the foot end of the frame 200 in order to accommodate occupant egress from the foot end. For example, in certain embodiments, the patient support apparatus 100 can be adjusted so that its profile mimics that of a chair.

The patient support apparatus 100 further includes a user interface 230 that can be used to control and adjust the various functions of the patient support apparatus 100. For example, the user interface 230 can includes input devices such as buttons, switches, and/or a touchscreen display to adjust the position of the frame 200, the firmness of the mattress 202, reset one or more alarms, and control other functions of the patient support apparatus 100. In the example shown in FIG. 2, the user interface 230 is positioned on the upper right siderail 214.

Additionally, the patient support apparatus 100 includes audio assembly 240 that has at least one speaker to provide audio instructions to the patient P. For example, the audio assembly 240 and speaker can be used to provide audio instructions for the patient P to self-turn when the patient P is able to do so. Also, the audio assembly 240 may include a microphone that can enable two-way audio communication between the patient P and the caregivers.

In some examples, one or more of the siderails 210-216 are provided with patient strength sensors 242. Instructions can be provided through the audio assembly 240 for the patient P to squeeze the patient strength sensors 242 for assessing patient P′s strength remotely. The data from the patient strength sensors 242 is sent to the controller 102, and can be combined with the aggregated movement data to determine the patient P′s strength and mobility. The strength data from the patient strength sensors 242 can be transmitted to the network 20 for storing in the patient P′s EMR 142, or for displaying on the mobile devices 130 or nurses' station 160.

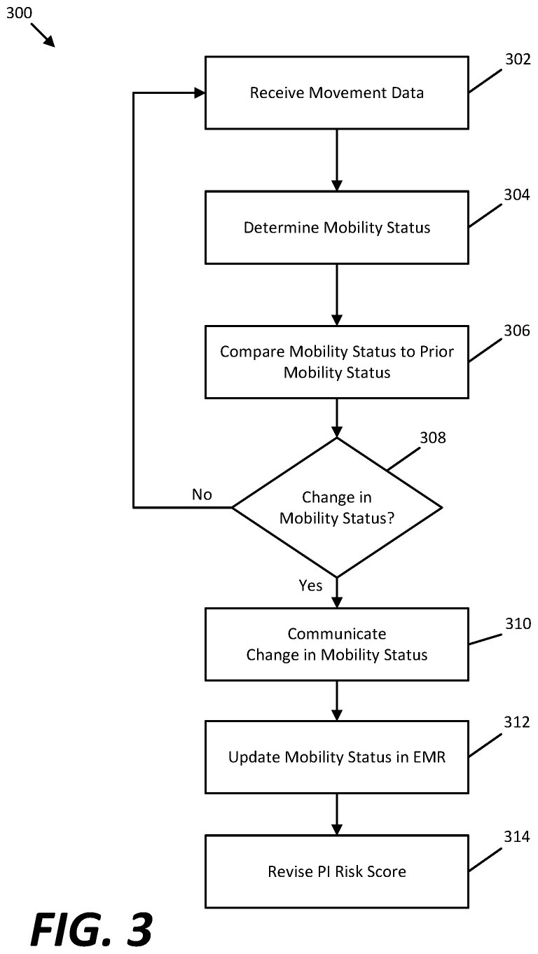

FIG. 3 illustrates a method 300 of monitoring and communicating a mobility status of the patient P. The method 300 can be performed by the controller 102. In some examples, the method 300 is enabled based on the risk scores for falls and pressure injuries. For example, the method 300 is performed when the patient P has a moderate or high risk for falls and pressure injuries, and is not performed when the patient P has a low risk for falls and pressure injuries. Also, the method 300 can be performed when data acquired from the patient P′s EMR 142 indicates that the patient P is at risk for falls or pressure injuries such as due to prescribed medications that can cause the patient P to be lethargic and have reduced mobility.

The method 300 includes an operation 302 of receiving movement data of the patient P. The movement data includes movement data collected from the patient support apparatus 100 and mobility detection devices 120 within the patient environment 12. As described above, the patient support apparatus 100 includes sensors 204 that detect movement of the patient P while the patient P rests on the patient support apparatus 100, and the mobility detection devices 120 each include one or more sensors that detect movements of the patient P in the patient environment 12 when the patient P is not resting on the patient support apparatus 100.

Next, the method 300 includes an operation 304 of determining a mobility status of the patient P based on at least the movement data received in operation 302. As described above, the controller 102 can include one or more algorithms that use the movement data as inputs to compute the mobility status of the patient P as an output. In some examples, in addition to using the movement data as inputs, the algorithms additionally use data received from the EMR 142 of the patient P, such as the patient P′s recorded vital signs including non-invasive blood pressure (NIBP), SpO₂, respiration rate, hear rate, temperature, and level of consciousness (LOC), to compute the mobility status of the patient P as an output. In some examples, the mobility status is scaled or converted to correspond to a bedside mobility assessment tool (BMAT) score, an activity measure for post-acute care (AM-PAC) score, or a proprietary mobility score.

Next, the method 300 includes an operation 306 of comparing the mobility status, determined from operation 304, with a prior mobility status stored in the EMR 142 of the patient P. As an illustrative example, the prior mobility status can be determined upon the patient P′s admission to the healthcare facility. As another illustrative example, the prior mobility status can be determined from a previous day (e.g., yesterday) or from an earlier time of the same day.

The method 300 determines at operation 308 whether the mobility status, determined from operation 304, differs from the prior mobility status stored in the EMR 142. When there is no difference between the mobility status determined from operation 304 and the prior mobility status stored in the EMR 142 (i.e., “No” at operation 308), the method 300 returns to operation 302 and continues to monitor the mobility status of the patient P. In some examples, before returning to operation 302, the method 300 can update a timestamp of the mobility status stored in the EMR 142 such as to confirm that the mobility status was recently updated.

When there is an inconsistency such that the mobility status determined from operation 304 differs from the prior mobility statues stored in the EMR 142 (i.e., “Yes” at operation 308), the method 300 proceeds to an operation 310 of communicating the change in mobility status to a caregiver or family member. Communicating the change in mobility status can help alert caregivers to possible deterioration of the patient P′s condition that may impact need for additional equipment or further evaluation.

The change in mobility status can be communicated via a notification sent to the mobile device 130 of a caregiver or family member. The notification can be displayed on the lock screen of the mobile device 130 to alert the caregiver or family member of the changed mobility status of the patient P. Alternatively, a text message that indicates the changed mobility status can be sent to the mobile device 130 of the caregiver or family member. The notification and/or text message can be generated by the controller 102 and sent to the mobile device 130 via the network 20 upon detecting the change in the patient P′s mobility status.

In some examples, the method 300 can include an operation 312 of updating the mobility status in the EMR 142. In some examples, the controller 102 automatically updates the mobility status of the patient P stored in the EMR 142 via the network 20. Alternatively, the controller 102 can prompt a caregiver to update the mobility status stored in the EMR 142.

The mobility status for patient P can be used to adjust a care protocol for the patient P from “self-turn” to “assisted-turn”. While these care protocols will be described in more detail, self-turn means that the patient P can turn their body to avoid a pressure injury without the need for assistance, and assisted-turn means that the patient P cannot turn their body unless they are assisted by someone else such as a caregiver or family member.

In some further examples, the method 300 can also include a further operation 314 of revising the pressure injury risk score for the patient P based on at least the updated mobility status. The pressure injury risk score can be based on the Braden Scale, the CMUNRO Scale, the Scott Triggers Tool, and similar scales and tools. The pressure injury risk score is inversely proportional to the patient P′s mobility. For example, when the mobility of the patient P decreases, the pressure injury risk score increases because the patient P moves their body less frequently. Conversely, when the mobility of the patient P increases, the pressure injury risk score decreases because the patient P moves their body more frequently.

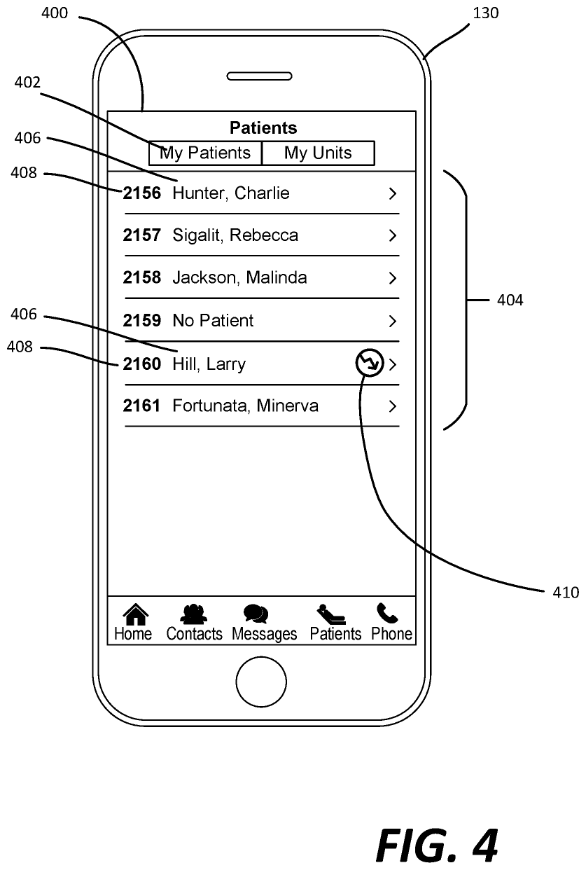

FIG. 4 illustrates a user interface 400 that can be generated on a mobile device 130 in response to a detected change in the patient P′s mobility status. The user interface 400 displays a “My Patients” tab 402 that includes a summary 404 of patients assigned to or associated with a caregiver. For each patient listed in the summary 404, identification information such as the patient's name 406 and room number 408 are provided. An icon 410 is displayed to indicate a change in the mobility status of the patient P. In this example, the icon 410 is an arrow pointing downward and colored red to indicate that the mobility status of the patient P has decreased.

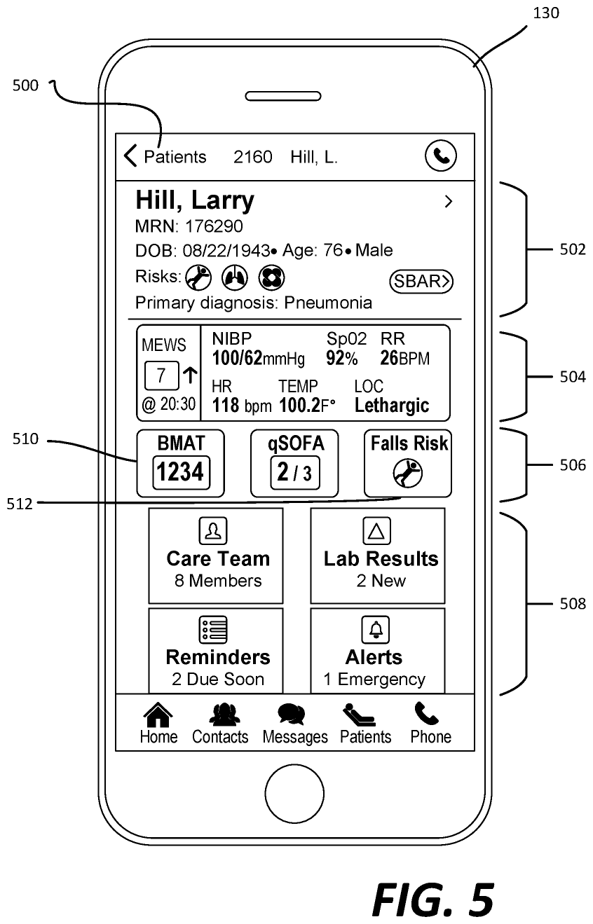

FIG. 5 illustrates another user interface 500 that can be generated on a mobile device 130 upon selection of the patient P from the summary 404 provided in the user interface 400 of FIG. 4. The user interface 500 displays information from the patient P′s EMR 142. The network 20 can integrate the mobile device 130 with the EMR server 140 and controller 102 such that the user interface 500 displays data acquired from the controller 102 and the patient P′s EMR 142.

The user interface 500 includes a bibliographic section 502 that identifies the patient P′s name (e.g., “Hill, Larry”), medical record number (MRN) (e.g., “MRN: 176290”), date of birth (e.g., “DOB: Aug. 22, 1943”), age (e.g., “Age: 76”), sex (e.g., “Male”), identified risks (e.g., falls risk, pneumonia risk, injury risk, etc.), and primary diagnosis (e.g., “pneumonia”).

The user interface 500 includes a vital signs dashboard 504 that can display the patient P′s recorded vital signs such as non-invasive blood pressure (NIBP), SpO₂, respiration rate, hear rate, temperature, and level of consciousness (LOC) (e.g., “lethargic”). Additionally, the vital signs dashboard 504 can display a modified early warning score (MEWS) assigned to the patient P that can include an arrow icon to indicate whether it is trending upwards or downwards, and a time stamp to indicate the last time it was updated.

The user interface 500 further includes a risk dashboard 506 that includes a bedside mobility assessment tool (BMAT) score 510. In certain examples, the BMAT score 510 is determined and continuously updated by the controller 102 based on the movement data collected from the sensors on the patient support apparatus 100 and mobility detection devices 120 located in the patient environment 12. The mobility status determined by the controller 102 can be scaled or otherwise converted into the BMAT score 510. Advantageously, the BMAT score 510 is objectively calculated by the controller 102 such that human subjectivity is removed from the calculation of the BMAT score 510, thereby enhancing the accuracy of the score.

The user interface 500 can further include a quick sequential organ failure assessment, or alternatively, a quick sepsis-related organ failure assessment score (qSOFA), and a falls risk icon 512 that may change color based on the patient P′s falls risk. The risk dashboard 506 may include additional types of risk scores based on the patient P′s condition.

The user interface 500 can further include a care communication section 508 that includes selectable icons that when selected display additional information related to the care of the patient P. For example, the selectable icons can include icons that when selected display the caregivers and care team members assigned to the patient P, the patient P′s lab results, reminders on care protocols for the patient P, and alerts generated for the patient P.

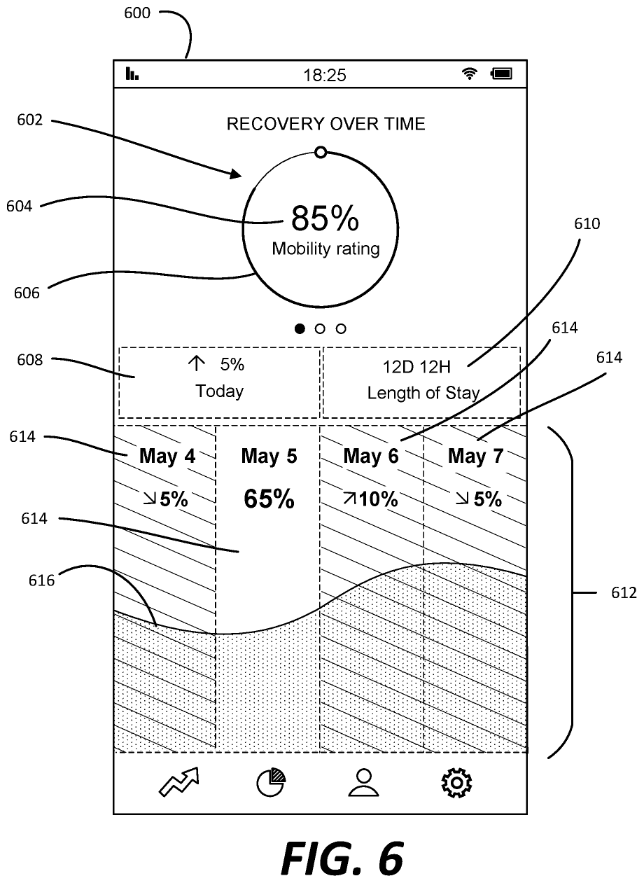

FIG. 6 illustrates another user interface 600 that can be generated on a mobile device 130. The user interface 600 can be generated during operation 310 of the method 300 for communicating the change in mobility status to a caregiver or family member. The user interface 600 displays a mobility status 602 of the patient P that is determined and continuously updated by the controller 102 based on the movement data collected from the sensors on the patient support apparatus 100 and the mobility detection devices 120 in the patient environment 12. Advantageously, the user interface 600 displays the mobility status 602 of the patient P as it changes in real-time, and in an easily understood format that help guide care practice.

The mobility status 602 includes a score 604 that indicates the mobility status of the patient P. In the illustrative example provided in FIG. 6, the score 604 is a numerical percentage on a scale of 0-100% in which 0% indicates that the patient P is not mobile, and 100% indicates that the patient P is highly mobile. Additional types of scores and scales are possible for the user interface 600 to indicate the mobility status of the patient P.

Additionally, the mobility status 602 can include icon 606 that visually depicts the mobility status of the patient P. As an example, the icon 606 can be a circle that is partially filled based on the score 604. In the illustrative example provided in FIG. 6, the score 604 is 85% such that the circle is filled by about 85% to provide a visual depiction of the mobility status of the patient P. Additionally, the icon 606 can change color to further enhance the visual depiction of the mobility status. For example, the icon 606 can be colored red to indicate low mobility for the patient P based on the score 604, and can gradually shift from red to yellow and from yellow to green to indicate higher mobility for the patient P based on the changes in the score 604.

The user interface 600 includes a daily trend section 608 that indicates a change in the mobility status of the patient P for the present day. In the example provided in FIG. 6, the daily trend section 608 indicates that the mobility of the patient P has increased by 5% today, and includes an arrow pointing upwards to indicate the mobility of the patient P is trending upwards. In some examples, the arrow is colored green or blue to indicate the upward trend. In examples where the mobility of the patient P has decreased, the arrow will point downwards and can be colored red or yellow to indicate the downward trend in the patient P′s mobility.

The user interface 600 can include a duration section 610 that indicates the patient P′s length of stay in the healthcare facility. The duration section 610 can be displayed next to the daily trend section 608. The duration section 610 can include the number of days and/or hours of the patient P′s length of stay. In the illustrative example provided in FIG. 6, duration section 610 indicates that the patient P has been in the healthcare facility for 12 days and 12 hours.

The user interface 600 includes a trending section 612 that visually depicts changes in the patient P′s mobility over the course of several days (e.g., three, four, five, six, or seven days). The trending section 612 includes a curve 616 that visual depicts the patient P′s mobility. The curve 616 is divided by day to indicate daily changes in the patient P′s mobility.

In the illustrative example provided in FIG. 6, the trending section 612 indicates that the patient P had a 5% decrease in their mobility on May 4, a 10% increase in their mobility on May 6, and a 5% decrease in their mobility on May 7. The daily decreases and increases in the patient P′s mobility can be visually depicted by arrows displayed above the curve 616 that point downwardly or upwardly, respectively. The arrows can also be color coded such as by having a red color to depict a decrease in daily mobility and a green color to depict an increase in daily mobility. The mobility trends displayed in the trending section 612 can help caregivers identify patient improvement and readiness for discharge, or possible delays for patient discharge.

A day within the trending section 612 is selectable for displaying additional information with respect to the mobility status of patient P for that day. For example, when a day is selected within the trending section 612, the mobility score of the patient P is displayed for that day. In the example provided in FIG. 6, May 5 is shown as selected. In this illustrative example, the trending section 612 depicts that the patient P had a score of 65% on May 5.

Additional metrics can also be displayed in the user interface 600 such as a visualization of the number and frequency of turns (whether self-turns or assisted-turns), and the amount of time that the patient P has spent out of the patient support apparatus 100. The visualizations and scores displayed on the user interface 600 can be provided to caregivers across different units of the healthcare facility where the patient P is admitted.

Advantageously, the user interface 600 can help augment clinical decision making, reduce cognitive burden, and simplify care coordination for the caregivers responsible for caring for the patient P. For example, the user interface 600 can help caregivers determine whether the patient P is advancing toward mobility independence, whether the patient P should receive a physical therapy consultation, whether the patient P has returned to baseline mobility such as after a surgical operation, and whether the patient P is ready for discharge.

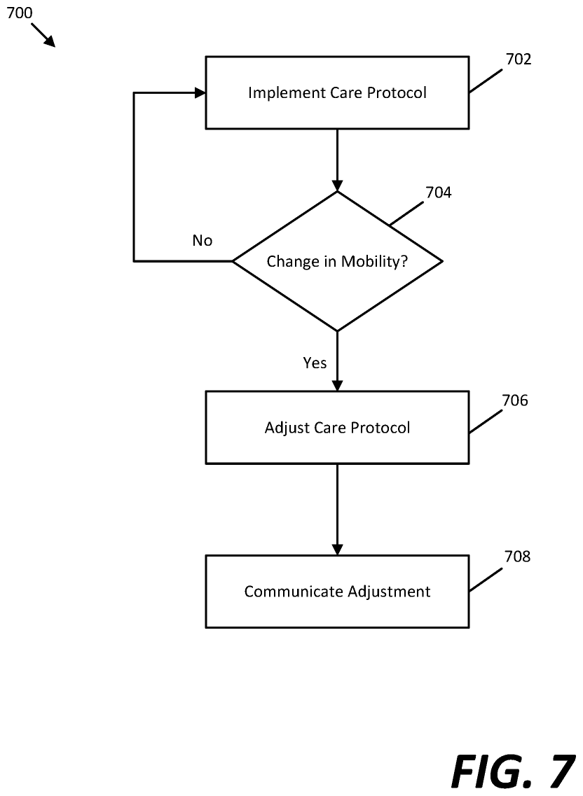

FIG. 7 illustrates a method 700 of adjusting a care protocol for the patient P based on a changed mobility status of the patient P. The method 700 can be performed by the controller 102. The method 700 includes an operation 702 of implementing a care protocol. Certain care protocols, such as physical therapy, can be ordered to improve the patient P′s mobility, and thereby decrease the patient P′s length of stay in the healthcare facility. Other types of care protocols can be implemented to reduce patient injuries, and can thereby also decrease the patient P′s length of stay in the healthcare facility and improve discharge planning.

One illustrative example of a care protocol is a turn protocol to prevent pressure injuries. Pressure injuries, also called pressure ulcers, decubitus ulcers, and bedsores, are injuries to skin and underlying tissue resulting from prolonged pressure on the skin that occurs due to a patient having limited movement while in bed. When a patient is unable to turn their body without assistance, such as when a patient is in a coma, the patient is designated as “assisted-turn” and the turn protocol requires a caregiver to periodically turn the patient's body to avoid pressure injuries. When the patient is able to turn their body without assistance, the patient is designated as “self-turn” and the turn protocol may periodically send a reminder to the patient or caregiver that notifies of the need to turn the patient's body to avoid pressure injuries.

The patient P may be erroneously designated as “self-turn” even though the patient P is not able to turn their body without assistance. This may occur when the patient P′s mobility deteriorates during their stay in the healthcare facility such that the patient P was initially able to self-turn, but is no longer able to do so because of their deteriorated condition. Also, the patient P may become unable to self-turn due to sedation or due to taking other medications. Additionally, the patient P may be physically able to self-turn to prevent pressure injuries, but is non-compliant to instructions to self-turn or does not understand the instructions to self-turn.

The method 700 next includes an operation 704 of determining whether the patient P has experienced a change in mobility status. As described above, the mobility status of the patient P is determined and continuously updated by the controller 102 based on the movement data collected from the sensors on the patient support apparatus 100 and mobility detection devices 120 in the patient environment 12. The mobility status, including trends and changes in the mobility status, can be displayed in a user interface such as the user interface 600 of FIG. 6.

When there is no change in mobility status (i.e., “No” at operation 704), the method 700 returns to operation 702 and continues to implement the care protocol. When there is a change in mobility status is detected (i.e., “Yes” at operation 704), the method 700 proceeds to an operation 706 of adjusting the care protocol based on the changed mobility status.

In some examples, when the care protocol is a turn protocol designated as “self-turn” at operation 702, operation 706 changes the turn protocol to “assisted-turn” when the mobility status of the patient P changes such that the patient P is no longer able to self-turn. As another example, when the care protocol is a turn protocol designated as “assisted-turn” at operation 702, operation 706 changes the turn protocol to “self-turn” when the mobility status of the patient P changes such that the patient P is now able to self-turn without assistance.

As another example, operation 706 can include ordering a physical therapy consultation based on the changes in the mobility status of the patient P. For example, when the mobility status of the patient P increases beyond a certain threshold, a physical therapy consultation can be automatically ordered under operation 706 due to the patient P being physically strong enough to begin physical therapy. As another example, when the mobility status of the patient P decreases, a physical therapy consultation can be automatically ordered under operation 706 to prevent further deterioration of the patient P′s mobility status. Thus, the method 700, when implemented by the controller 102, can reduce the delay in ordering physical therapy, and thereby decrease the patient P′s length of stay in the healthcare facility.

As another example, operation 706 can include making changes to the patient P′s discharge plan based on the change in mobility status detected in operation 704. For example, when the patient P′s mobility status decreases, the discharge date for the patient P to be discharged from the healthcare facility can be delayed. Alternatively, when the patient P′s mobility status increases, the discharge date for the patient P can be adjusted to be earlier. In some examples, operation 706 can include communicating the changes to the patient P′s discharge plan to an admission/discharge/transfer (ADT) server of the healthcare facility.

In some examples, operation 706 automatically adjusts the care protocol and stores the changes to the care protocol in the EMR 142 of the patient P. Alternatively, in other examples, operation 706 includes sending a notification to a caregiver requesting the caregiver to manually change the care protocol based on the change in mobility status of the patient P. This alternative example can allow caregivers to use their professional judgement to determine whether the care protocol should be changed based on the change in mobility status.

In some examples, the method 700 includes an operation 708 of communicating the change in the care protocol to a caregiver or family member. For example, the change in the care protocol can be communicated via a notification sent to the mobile device 130 of a caregiver or family member. Alternatively, a text message that indicates the change in the care protocol can be sent to the mobile device 130 of the caregiver or family member. The notification and/or text message that identifies the change in the care protocol can be generated by the controller 102 and sent by the network 20 to the mobile device 130.

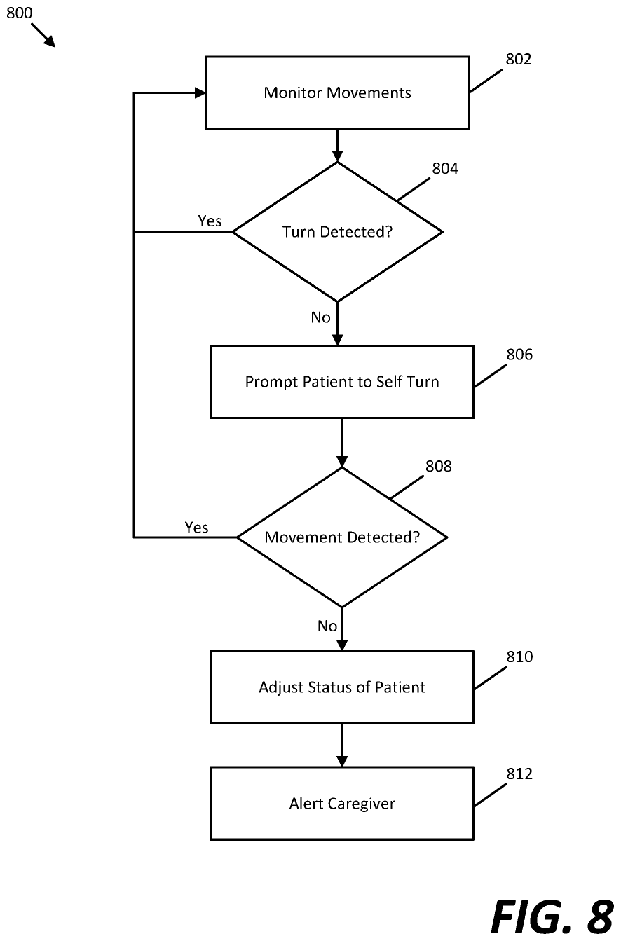

FIG. 8 illustrates a method 800 of turning a patient to mitigate pressure injuries. The method 800 can be performed by the controller 102. In certain examples, the method 800 is enabled and disabled based on the patient P′s mobility status stored in the patient P′s EMR 142. For example, the method 800 can be disabled when the patient P is designated as “assisted-turn.” As another example, the method 800 can be disabled when the patient P is highly mobile such that the patient P does not need to be turned to avoid pressure injuries. As another example, the method 800 can be disabled when it is detected that the patient P is sleeping. The method 800 can be performed by the controller 102 when the patient environment 12 is a patient room within the healthcare facility, or is an emergency department or PreOp area of the healthcare facility where the patient P can wait for long periods of time on a bed or stretcher.

The method 800 includes an operation 802 of monitoring the movement of the patient P in the patient environment 12. As described above, the movements of the patient P are monitored and continuously updated by the controller 102 based on the movement data collected from the sensors on the patient support apparatus 100 and mobility detection devices 120 located in the patient environment 12. Additionally, the movements of the patient P, including trends and changes in the mobility status, can be displayed in a user interface such as the user interface 600.

Next, the method 800 includes an operation 804 of detecting whether the patient P has turned their body within a predetermined time period. For example, operation 802 can detect whether the patient has turned their body from the right side to the left side, or from left side to right side within the predetermined period of time to avoid a pressure injury.

The predetermined period of time may vary based on the patient P′s risk for pressure injury which may be designated and stored in the patient P′s EMR 142. As an illustrative example, when the patient P has a high risk for pressure injury, the predetermined period of time can be set for every hour, when the patient P has a moderate risk for pressure injury, the predetermined period of time can be set for every two hours, and when the patient P is has a low risk for pressure injury, the predetermined period of time can be set for every three hours.

When it is detected that the patient P has turned their body within the predetermined period of time (i.e., “Yes” at operation 804), the method 800 returns to operation 802 and continues to monitor the movements of the patient P. When it is detected that the patient P has not turned their body within the predetermined period of time (i.e., “No” at operation 804), the method 800 proceeds to an operation 806 of prompting the patient P to self-turn.

Operation 806 can be performed by providing audio instructions to the patient P through the audio assembly 240 of the patient support apparatus 100, shown in FIG. 2. The audio instructions can encourage the patient P to self-turn which can advantageously free up more time for the caregivers and more actively engage the patient P in their own care. The voice prompt can be a pre-recorded message such as “You have not moved in the last hour. Please shift your position.” Alternatively, the voice prompt can be a message from a caregiver or family member that encourages the patient P to reposition and turn their body. A message from a family member can help support patient and family engagement.

Next, the method 800 proceeds to an operation 808 of detecting whether the patient P has turned their body within a predetermined time period after the audio instructions have been provided. Operation 808 can be similar to operation 804. The predetermined time period under operation 808 is shorter than the predetermined time period under operation 804. For example, the predetermined time period under operation 808 may be set for 5, 10, 15, 20, or 30 minutes after the audio instructions have been provided to the patient P.

When it is detected that the patient P has turned their body within the predetermined period of time (i.e., “Yes” at operation 808), the method 800 returns to operation 802 and continues to monitor the movements of the patient P. When it is detected that the patient P has not turned their body within the predetermined period of time (i.e., “No” at operation 808), the method 800 proceeds to an operation 810 of adjusting the status of the patient P. For example, operation 810 can include changing the status from self-turn to assisted-turn.

Next, the method 800 proceeds to an operation 812 of alerting a caregiver or family member of the adjusted status of the patient P. The adjusted status can be communicated via a notification or text message sent to the mobile device 130 of a caregiver or family member. The notification and text message that identifies the adjusted status of the patient P can be generated by the controller 102 and sent by the network 20 to the mobile device 130 of the caregiver or family member. Also, the adjusted status can be communicated to the nurses' station 160.

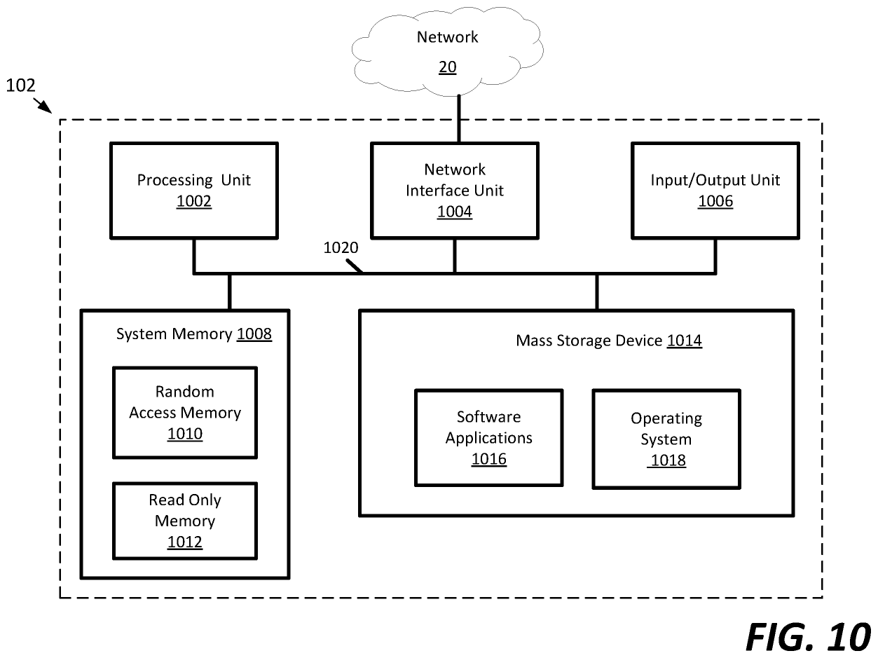

FIG. 10 schematically illustrates the controller 102 used to implement aspects of the present disclosure. While the controller 102 is shown in FIG. 10, other computing devices that are part of the mobility management system 10 can have similar components.

The controller 102 has a processing unit 1002, a system memory 1008, and a system bus 1020 coupling the system memory 1008 to the processing unit 1002. The processing unit 1002 is an example of a processing device such as a central processing unit (CPU).

The system memory 1008 is an example of a computer readable data storage device. The system memory 1008 includes a random-access memory (“RAM”) 1010 and a read-only memory (“ROM”) 1012. Input/output logic containing the routines to transfer data between elements within the controller 102, such as during startup, is stored in the ROM 1012.

The controller 102 can also include a mass storage device 1014 that is able to store software instructions and data. The mass storage device 1014 is connected to the processing unit 1002 through a mass storage controller (not shown) connected to the system bus 1020. The mass storage device 1014 and its associated computer-readable data storage medium provide non-volatile, non-transitory storage for the controller 102.

Although the description of computer-readable data storage media contained herein refers to a mass storage device, it should be appreciated by those skilled in the art that computer-readable data storage media can be any available non-transitory, physical device or article of manufacture from which the device can read data and/or instructions. The mass storage device 1014 is an example of a computer-readable storage device.

Computer-readable data storage media include volatile and non-volatile, removable and non-removable media implemented in any method or technology for storage of information such as computer-readable software instructions, data structures, program modules or other data. Example types of computer-readable data storage media include, but are not limited to, RAM, ROM, EPROM, EEPROM, flash memory or other solid-state memory technology, or any other medium which can be used to store information, and which can be accessed by the device.

The controller 102 may operate in a networked environment using logical connections to remote network devices, including the mobile devices 130, EMR server 140, nurse call server 150, and nurses' station 160, through the network 20. The controller 102 connects to the network 20 through a network interface unit 1004 connected to the system bus 1020. The network interface unit 1004 may also be utilized to connect to other types of networks and remote computing systems.

The controller 102 can also include an input/output controller 1006 for receiving and processing input from a number of input devices. Similarly, the input/output controller 1006 may provide output to a number of output devices.

The mass storage device 1014 and the RAM 1010 can store software instructions and data. The software instructions can include an operating system 1018 suitable for controlling the operation of the device. The mass storage device 1014 and/or the RAM 1010 also store software instructions 1016, that when executed by the processing unit 1002, cause the device to provide the functionalities discussed in this document.

The various embodiments described above are provided by way of illustration only and should not be construed to be limiting in any way. Various modifications can be made to the embodiments described above without departing from the true spirit and scope of the disclosure.

What is claimed is:

1. A mobility management system comprising:
a controller having at least one processing device, and at least one computer readable data storage device storing software instructions that, when executed by the at least one processing device, cause the controller to:
receive movement data from sensors that detect movements of a patient resting in a patient support apparatus;
receive movement data from one or more mobility detection devices that detect movement of the patient when the patient exits the patient support apparatus;
determine a mobility status based on at least the movement data received from the patient support apparatus and the one or more mobility detection devices; and
adjust a care protocol for the patient based on the mobility status.

2. The system of claim 1, wherein the care protocol is adjusted from self-turn to assisted-turn.

3. The system of claim 1, wherein the care protocol is adjusted from assisted-turn to self-turn.

4. The system of claim 1, wherein the software instructions, when executed by the processing device, further cause the controller to:
display the mobility status on a mobile device.

5. The system of claim 1, wherein the software instructions, when executed by the at least one processing device, further cause the controller to:
store the mobility status to an electronic medical record.

6. The system of claim 1, wherein the software instructions, when executed by the at least one processing device, further cause the controller to:
revise a pressure injury risk score based on the mobility status.

7. The system of claim 1, wherein the software instructions, when executed by the at least one processing device, further cause the controller to:
prompt the patient to self-turn;
detect whether the patient turns within a predetermined time period; and
adjust the care protocol from self-turn to assisted-turn upon detection that the patient has not turned within the predetermined time period.

8. The system of claim 1, further comprising the one or more mobility detection devices, and the one or more mobility detection devices including at least one of a walk assist device, a wearable device, and a mounted device.

9. The system of claim 1, further comprising a walk assist device having one or more additional sensors that measure the patient's frequency of using the walk assist device, gait, speed, acceleration, number of steps, equilibrium, and wait times between movements.

10. The system of claim 1, wherein the controller aggregates the movements detected by the one or more mobility detection devices with the movements detected by the sensors in the patient support apparatus.

11. A method of determining a mobility status of a patient, the method comprising:
receiving a first set of movement data from sensors on a patient support apparatus;
receiving a second set of movement data from one or more mobility detection devices located within a patient environment around the patient support apparatus;
determining a mobility status based on the first and second sets of movement data;
comparing the mobility status to a prior mobility status; and
communicating the mobility status to a mobile device.

12. The method of claim 11, further comprising:
storing the mobility status to an electronic medical record.

13. The method of claim 11, further comprising:
adjusting a care protocol based on the mobility status.

14. The method of claim 11, further comprising:
revising a pressure injury risk score based on the mobility status.

15. The method of claim 11, further comprising:
adjusting a care protocol from self-turn to assisted-turn based on the mobility status.

16. A computer-readable data storage medium comprising software instructions that, when executed, cause at least one computing device to:
receive a first set of movement data from sensors on a patient support apparatus;
receive a second set of movement data from one or more mobility detection devices located within a patient environment around the patient support apparatus;
determine a mobility status based on the first and second sets of movement data;
compare the mobility status to a prior mobility status; and
communicate the mobility status to a mobile device.

17. The computer-readable data storage medium of claim 16, wherein the software instructions further cause the at least one computing device to:
store the mobility status to an electronic medical record.

18. The computer-readable data storage medium of claim 16, wherein the software instructions further cause the at least one computing device to:
adjust a care protocol based on the mobility status.

19. The computer-readable data storage medium of claim 16, wherein the software instructions further cause the at least one computing device to:
revise a pressure injury risk score based on the mobility status.

20. The computer-readable data storage medium of claim 16, wherein the software instructions further cause the at least one computing device to:
adjust a care protocol from self-turn to assisted-turn based on the mobility status.
